(19) United States
(12) Patent Application Publication
(10) Pub. No.: US 2026/0007539 A1
(43) Pub. Date: Jan. 8, 2026

(54) AN INSERTION AND WITHDRAWAL SYSTEM FOR INSERTING, SUBTRACTING, AND CONTAINING AN OSTOMY DEVICE

(71) Applicant: AMPA MEDICAL APS, København N (DK)

(72) Inventors: Marie Filippson PARSLOV, København N (DK); Cecilie AMMITZBØLL, København N (DK); Christoffer Dupont SKOVSBY, København N (DK)

(73) Assignee: AMPA MEDICAL APS, København N (DK)

(21) Appl. No.: 18/874,749

(22) PCT Filed: Jun. 15, 2023
(86) PCT No.: PCT/EP2023/066122

§ 371 Date: Dec. 13, 2024

(30) Foreign Application Priority Data
Jun. 16, 2022 (DK) PA 2022 70325
May 4, 2023 (DK) PA 2023 70221

(51) Int. Cl. A61F 5/443; A61F 5/44

(52) U.S. Cl. CPC A61F 5/443; A61F 5/4401

(57) ABSTRACT

An insertion and withdrawal system for insertion of an ostomy device through an ostomy of a human being, subtracting the ostomy device, and containing the ostomy device during and after subtraction, the insertion and withdrawal system including the ostomy device, a protection shield, a holster attached to the protection shield, and one or more withdrawers. A protection shield and a holster for use in an insertion and withdrawal system

### TECHNICAL FIELD

The invention relates to an insertion and withdrawal system for insertion of an ostomy device through an ostomy of a human being, subtracting the ostomy device, and containing the ostomy device upon, during, and after subtraction, the insertion and withdrawal system comprising the ostomy device, a protection shield, a holster attached to the protection shield, and one or more withdrawal means.

### BACKGROUND ART

An ileostomy is an artificial intestinal opening where the large intestine (colon) is surgically removed due to illness or trauma and the small intestine (ileum) is redirected through the abdominal wall and sutured to the skin, constituting the ileostomy. Hereafter the intestinal content is emptied through the ileostomy. Ileostomies do not have a sphincter to control voluntary excretion of intestinal content and therefore the ileostomy is incontinent with continuously and uncontrollable excretion of intestinal content. For the majority of people with ileostomies the intestinal content is liquid and the volume is 500-1500 mL/day.

Beside ileostomies, other ostomies are created, such as colostomies (colon) or urostomies (urinary tract). Ileostomy defines an ostomy from the ileum, but is also commonly used to define ostomies from the small intestine in general, thereby also covering jejunostomies (jejunum). All the above mentioned may generally be known as ostomies.

The intestinal content from an ileostomy is collected in a stoma bag attached to a person's stomach by an adhesive plate. The stoma bag was invented in the 1950's and has since then been used to collect the intestinal output from an ileostomy. However, the stoma bag has considerable disadvantages.

First of all, the changed body image that comes with having a bag with intestinal content hanging from the abdomen is a psychological strain for most persons. Secondly, since a person with an ileostomy lacks the ability to hold back intestinal content, and the excretion of intestinal content into the stoma bag cause considerable noise during bowel movement, the user is often considerably socially handicapped—even when stoma bags are used properly. The adhesively attached stoma bags moreover have the disadvantage that the weight of the more or less filled bag causes discomfort and may result in the bag becoming involuntarily detached from the user's skin, resulting in leakage of intestinal contents.

Further, natural folding of the skin surrounding the stoma can cause difficulties in keeping the adhesive plate tight from leakage.

Moreover, users of stoma bags often get skin complications requiring medical treatment, due to skin contact with intestinal content and the aggressive adhesive used on a stoma plate to withstand the weight of the stoma bag.

Finally, physical exercise is complicated by the external storage of intestinal contents in a bag. Upon pushing, squeezing or clamping the bag there is an increased risk of leakage.

People with an ileostomy therefore demand alternative solutions for the stoma bag but so far there are no functional alternatives on the market. Solutions that can withhold solid stool in a colostomy (stoma of the large intestine) exist, but these solutions do not work in an ileostomy because of the large volumes of liquid content that ileum produce, which existing solutions do not manage to seal properly.

There is thus a need of another type of system to collect the liquid of the intestine while also forming a closure hereof. In particular, there is a need of a closure that is suited for the small intestine (ileum) and form a closure hereof, and with the ability to retain the majority of that liquid. There is further a need for such a type of system to be hygienical and easy to handle, also on the move.

### SUMMARY

The present invention solves the above problems by providing an integrated shield and sleeve system attached to an intestinal ostomy device; hence, disclosed herein in a first aspect is an insertion and withdrawal system for insertion of an ostomy device through an ostomy of a human being, subtracting the ostomy device, and containing the ostomy device after subtraction, the insertion and withdrawal system comprising: the ostomy device having an expandable elongated body comprising one or more absorbent materials; a protection shield having a first side and a second side; a holster attached to the protection shield, the holster configured to be in a folded state prior to insertion of the ostomy device through the ostomy of the human being; and one or more withdrawal means; wherein the first side of the protection shield has one or more attachment means for attaching the protection shield to the human being; wherein the one or more absorbent materials is configured to absorb intestinal fluid and waste inside the intestines of the human being; wherein the ostomy device is configured to be pulled out of the intestines of the human being by means of the one or more withdrawal means; wherein the holster is configured to transition to an unfolded state hereby covering the ostomy device when the ostomy device is pulled out of the intestines of the human being by means of the one or more withdrawal means; and wherein the holster in the unfolded state is further configured to contain the ostomy device after subtraction.

Hereby is provided a system, which is not only easy to use (as it is a potential all in one product), and being hygienical to insert, but it is also effective in absorbing and containing the intestinal liquid inside the intestine upon usage of the ostomy device, thus normalizing body image by getting rid of the stoma bag, replaced by a small protective shield over the stoma. Further, due to the internal absorption of intestinal waste and the protective shield, leakage may be avoided by providing a good closure of the ostomy. Further, due to the protective shield, secretion or moist from the ostomy may be captured by an integrated padding in the protective shield. The protective shield also protects the ostomy from drying out or be damaged. Moreover, by avoiding skin contact with intestinal content, and by avoiding the aggressive adhesive used on stoma plates, skin complications may be avoided.

Further, the system makes it hygienical to remove the ostomy device, when the device has absorbed the intestinal liquids, by simply pulling the withdrawal means and the holster will wrap around the used ostomy device, hereby avoiding contact with the fingers or the skin of the person pulling the withdrawal means. The ostomy device can then be disposed in the holster. The holster is configured such that it unfolds during subtraction of the ostomy device, hereby covering the ostomy device upon, during, and after subtraction of said device. The holster may be configured to unfold during subtraction of the ostomy device via an elastic expansion, such that the holster is configured to unfold and elastically expand during subtraction of the ostomy device. Further, the system as disclosed herein is minimising the amount of noise created, as there will be no excretion of intestinal content into a stoma bag, which cause considerably noise during bowel movement.

Disclosed herein in a second aspect is a protection shield for use in an insertion and withdrawal system according to the first aspect, the protection shield having a first side and a second side.

Disclosed herein in a third aspect is a holster for use in an insertion and withdrawal system according to the first aspect, the holster being configured to be in a folded state prior to insertion of the ostomy device through the ostomy of the human being.

Disclosed herein in a fourth aspect is the use of an insertion and withdrawal system according to the first aspect for insertion of an ostomy device through an ostomy of a human being.

Disclosed herein in a fifth aspect is the use of an insertion and withdrawal system according to the first aspect for subtracting an ostomy device through an ostomy of a human being.

Disclosed herein in a sixth aspect is the use of an insertion and withdrawal system according to the first aspect for containing an ostomy device after subtraction of the ostomy device through the ostomy of a human being.

Disclosed herein in a seventh aspect is the use of a protection shield and/or a holster according to the first aspect for insertion of an ostomy device through an ostomy of a human being.

Disclosed herein in an eighth aspect is the use of a protection shield and/or a holster according to the first aspect for subtracting an ostomy device through an ostomy of a human being.

Disclosed herein in a ninth aspect is the use of a protection shield and/or a holster according to the first aspect for containing an ostomy device after subtraction of the ostomy device through the ostomy of a human being.

Disclosed herein in a tenth aspect is a method for insertion of an ostomy device through an ostomy of a human being, the method comprising the steps of:
providing the insertion and withdrawal system according to the first aspect;
placing the system at an opening surrounding the stoma, with the stoma being able to protrude inside the protection shield;
insert into the stoma opening, guided by a finger or alike, the ostomy device; and
removing the finger or alike and the ostomy device is in place with the one or more withdrawal means extending out from the stoma opening.

Disclosed herein in an eleventh aspect is a method for subtracting an ostomy device inserted through an ostomy of a human being, the method comprising the steps of:
withdrawal of the ostomy device according to the first aspect by pulling in the one or more withdrawal means, such that the ostomy device is pulled out through the stoma opening; and
simultaneously unfolding the holster by pulling the one or more withdrawal means and the ostomy device, such as pulling through the through hole or opening of the protection shield.

### BRIEF DESCRIPTION OF THE DRAWINGS

FIG. 1 shows various embodiments of the insertion and withdrawal system of the absorbent ostomy device prior to insertion.

FIG. 2 shows various embodiments of the insertion and withdrawal system of the absorbent ostomy device after insertion.

FIG. 3 shows various embodiments of the insertion and withdrawal system of the absorbent ostomy device after withdrawal.

FIG. 4 shows one embodiment of the insertion and withdrawal system of the absorbent ostomy device prior to insertion, in various perspectives and views.

FIG. 5 shows a cut through of the embodiment of FIG. 4.

FIG. 6 shows the embodiment of FIGS. 4 and 5 after insertion, in various perspectives and views.

FIG. 7 shows a cut through of the embodiment of FIG. 6.

FIG. 8 shows the embodiment of FIG. 4-7 after withdrawal, in various perspectives and views.

### DETAILED DESCRIPTION

The description herein of any aspect or embodiment of the invention using terms such as “comprising”, “having,” “including,” or “containing” with reference to an element or elements is intended to provide support for a similar aspect or embodiment of the invention that “consists of”, “consists essentially of”, or “substantially comprises” that particular element or elements, unless otherwise stated or clearly contradicted by context, e.g. a composition described herein as comprising a particular element should be understood as also describing a composition consisting of that element, unless otherwise stated or clearly contradicted by context. It will be further understood that the terms “comprises,” “comprising,” “includes” and/or “including,” when used in this specification, specify the presence of stated features, integers, steps, operations, elements, and/or components, but do not preclude the presence or addition of one or more other features, integers, steps, operations, elements, components, and/or groups thereof.

As used herein, the singular forms “a,” “an,” and “the” are intended to include the plural forms, including “at least one,” unless the content clearly indicates otherwise. “At least one” is not to be construed as limiting “a” or “an.”

Unless otherwise defined, all terms used herein (including technical and scientific terms) have the same meaning as commonly understood by those skilled in the art to which this invention pertains. It will be further understood that terms, such as those defined in commonly used dictionaries, should be interpreted as having a meaning that is consistent with their meaning in the context of the relevant art and will not be interpreted in an idealized or overly formal sense unless expressly so defined in the present specification.

The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

The present invention relates to an insertion and withdrawal system for insertion of an ostomy device through an ostomy of a human being, subtracting the ostomy device, and containing the ostomy device after subtraction. In other words, the present invention relates to an integrated protective shield and holster system attached to an ostomy device.

To remove an ostomy device in a hygienically manner the present invention discloses a system comprised of interconnected parts attached to the ostomy device.

Strings may be used as the one or more withdrawal means, and if they are attached to the end of the ostomy device, this may further protrude out from the end of the ostomy device penetrating the attached holster, or the holster is attached to the ostomy device just around the strings. In one or more embodiments, the string is in the form of a flat string, such as a flatwound string.

In one or more embodiments, the expandable elongated body extends along a longitudinal direction of the ostomy device, the expandable elongated body being configured to maintain a compressed state, having a first diameter along the longitudinal direction, prior to insertion of the ostomy device through the ostomy of the human being and configured to expand radially to a non-compressed state, having a second diameter along the longitudinal direction, after insertion into the intestines of the human being through the ostomy, wherein the second diameter is larger than the first diameter.

In one or more embodiments, the ostomy device is configured to absorb intestinal fluid and waste inside the intestines of the human being, the ostomy device comprising: an expandable elongated body extending along a longitudinal direction of the ostomy device and having a stoma end and an oral end spaced from the stoma end along the longitudinal direction, wherein the expandable elongated body comprises one or more absorbent materials; and one or more compression means configured to maintain the expandable elongated body in a compressed state, having a first diameter along the longitudinal direction, prior to insertion of the ostomy device through the ostomy of the human being; wherein the one or more withdrawal means are attached to the stoma end of the expandable elongated body; wherein the expandable elongated body is configured to expand radially to a non-compressed state, having a second diameter along the longitudinal direction, when released from the one or more compression means upon insertion into the intestines of the human being through the ostomy; and wherein the second diameter is larger than the first diameter.

The ostomy device may protrude through the protective shield while being covered by the holster which it is placed inside of.

In one or more embodiments, the protection shield is a band aid or a patch.

In one or more embodiments, the first side of the protection shield is configured to be in direct or indirect contact with the skin of the human being.

The outside of the protective shield may have a cutting in the surface material to make it possible for the ostomy device, covered by the holster, to be inserted and withdrawn through the cutting.

In one or more embodiments, the protection shield has a through hole extending from the first side to the second side.

In one or more embodiments, the through hole is circularly shaped.

In one or more embodiments, the holster is located in the through hole of the protection shield.

In one or more embodiments, one end of the holster is attached to the through hole and the holster is folded and placed around the through hole of the protection shield. In one or more embodiments, the holster is attached to the side of the through hole and harmonic folded. In one or more embodiments, the holster is attached to the second side of the protective shield and is harmonically folded from the outside towards the centre of the protective shield.

In one or more embodiments, one end of the holster is attached to the inside of the protection shield and the holster is folded and placed inside the protection shield.

In one or more embodiments, the holster is accessible from the second side of the protection shield.

One end of the holster may be attached to a protective shield in a circular manner around an opening (such as a through-hole) in the protective shield. The opening may be 3 to 6 cm in diameter. Thus, in one or more embodiments, the through hole has a diameter between 3 and 6 cm. Alternatively, one end of the holster may be attached to a protective shield in a circular manner around or close to the outside circumferential of the protective shield. The holster may then have an opening similar to the through hole of the protective shield, or it may extend outside being attached to an insertion sleeve, which holds the expandable elongated body prior to use. Alternatively, the thought hole in the holster may be attached to the insertion sleeve comprising the expandable elongated body prior to use.

Before usage, and when applied to the stoma, the system may be in a folded state with the holster circumferentially folded, such as in a ring like manner inside the protective shield, or on the outside of the protective shield, such as around an opening in the protective shield. In one or more embodiments, the holster is circumferentially folded in a harmonically shaped folding.

In one or more embodiments, the through hole is configured to allow the ostomy device to be inserted and withdrawn from the ostomy of the human being while the ostomy device is being covered by the holster. In one or more embodiments, the through hole comprises an insertion sleeve extending out and away from the through hole and the back side of the protective shield.

The backside of the protective shield—which is the side facing the skin, may have an adhesive around the opening to make it adhere to the skin. Thus, in one or more embodiments, the one or more attachment means for attaching the protection shield to the human being is an adhesive around the through hole to make the protection shield adhere to the skin of the human being. The adhesive may further be configured such that after the ostomy device has been pulled out of the intestine of the human being by means of the one or more withdrawal means, and the holster has transitioned to the unfolded state hereby covering the ostomy device, the protective shield can be folded at a middle section to close off the holster comprising the used ostomy device.

In one or more embodiments, the ostomy device protrudes through the through hole towards and past the first side of the protection shield while being covered by the holster on the second side of the protection shield. In one or more embodiment, the holster is attached to an insertion sleeve comprising the ostomy device on the second side of the protection shield prior to use. In one or more embodiments, the holster is both the insertion sleeve and the holster.

In one or more embodiments, the system further comprises a carbon filter. In one or more embodiments, the protective shield comprises one or more ventilation holes. In one or more embodiments, the one or more ventilation holes is one or more perforations in the protective shield. During digestion, foul-smelling gases are produced, which are usually emptied in a controlled manner with flatus. However, having a stoma such gases must be discharged continuously and unhindered to avoid accumulation of gas in the intestine. It is therefore an advantage of the present disclosure to have a protective shield comprising one or more ventilation holes, through which gas can be discharged. It is further an advantage if such ventilation holes are fitted with one or more filters, through which the gas has to pass prior to being discharged. It is therefore a further embodiment, that the one or more ventilation holes are fitted with one or more filters, such as charcoal/carbon filters, which remove odours from gasses passing through the ventilation holes.

In one or more embodiments, the protective shield comprises one or more integrated charcoal/carbon filters for odourless gas discharge.

In one or more embodiments, the carbon filter is incorporated into the protective shield.

In one or more embodiments, the carbon filter is incorporated into the protective shield in such a way that when the protective shield is attached to the user, the carbon filter will be positioned on the upper half, preferably the top part, of the protective shield. By positioning the filter in this way, the filter is removed from the exit of the stoma, and there is hence a less likelihood of the filter to get wet and hereby create a clutter at the filter, as the stoma may excrete small amount of secrete or faeces. If the filter is cluttered in this way, it will stop functioning properly. In one or more embodiments, the filter is positioned at or in the protective shield at the top part of the protective shield when in use. In one or more embodiments, the protective shield comprises a marking of the top part, such that the user knows how to position the protective shield on the stoma.

In one or more embodiments, the system further comprises a bandage for collecting secretion from the ostomy. In one or more embodiments, the system further comprises one or more integrated moisture absorbent pads. The one or more pads may be separate or integrated into the protective shield, such as surrounding the ventilation holes of the protective shield. In this way secretion from the outer part of the mucous membranes may be collected by an absorbent pad that is soft and conforms to the stoma. At the same time, the pad keeps the carbon filter, if present, dry so that it does not clog, and acts as a mechanical barrier between the carbon filter and the stoma.

In one or more embodiments, the holster is folded in a ring like manner in the folded state prior to insertion of the ostomy device through the ostomy of the human being.

In one or more embodiments, the protection shield, the holster, and the one or more withdrawal means are interconnected parts attached to the ostomy device.

The ostomy device may be attached in one end to a 20 to 80 cm long holster. Thus, in one or more embodiments, the holster is between 20 cm and 80 cm long in the unfolded state.

The holster may have a diameter of approximately 1.5 to 6 cm, however, the diameter can vary along the holster's length. Hence, in one or more embodiments, the holster has a diameter between 1.5 cm and 6 cm.

The protective shield may be a flat bag with a backside and an outside. In one or more embodiments, the protection shield is a flat bag having the first side being a backside or a skin side and the second side being an outside.

There may be a circular opening in the backside and the outside can be dome shaped to be able to contain a protruding stoma. In one or more embodiments, the second side is dome shaped hereby creating a cavity inside the protection shield between the first side and the second side to be able to contain a protruding stoma of the human being inside the cavity.

In one or more embodiments, the one or more withdrawal means is one or more strings attached to the ostomy device and extending through the holster on the second side of the protection shield.

The backside of the protective shield may hold additional attachment means to make it attachable to stoma baseplates. This could be adhesives connecting the protective shield to the stoma plate or different kinds of coupling or locking systems as e.g. click systems.

In one or more embodiments, the one or more attachment means is attachment means configured to be attachable to a stoma baseplate.

In one or more embodiments, the attachment means configured to be attachable to a stoma baseplate is a click system.

In one or more embodiments, the holster is made of plastic, silicone, latex, or combinations hereof. In one or more embodiments, the holster is made of a highly elastic material, such as a thermoplastic elastomer (TPE) or silicone. In one or more embodiments, the holster is made of a highly elastic material having a very low Young's modulus and an ability to stretch more than 800% before breaking. In one or more embodiments, the holster is made of a highly elastic thermoplastic elastomer having an ability to stretch more than 800% before breaking.

A very low Young's modulus could e.g. be a Young's modulus at or below 5 MPa, thus in one or more embodiments, the holster is made of a material having a Young's modulus at or below 5 MPa.

In one or more embodiments, the holster is made of a biodegradable material.

In one or more embodiments, the holster has a material thickness of at least 500 microns, such as at least 600 microns, or such as around 700 microns. The relatively large material thickness (500+microns) compared to typical film bags (30-50 microns), provides a closure having an effective odour barrier. This is both an advantage during use, but also after withdrawal, as this means that the product can be closed and thrown in the bin without any smell.

The holster and/or the insertion sleeve may be produced by blow moulding, dip moulding, or injection moulding.

In one or more embodiments, the system further comprise a collar attached to the second side and wherein the holster is attached to the collar.

In one or more embodiments, the holster is attached to the collar by connection means, such as elastic connection means.

The holster can be attached to the protective shield in other ways than on the backside. It can e.g. be attached to the inside or the outside material of the protective shield, just beneath the cutting. The holster can also have the full diameter of the protective shield, folded in the side seams of the protective shield hereby giving the protective shield some of the depth potentially needed to contain the protruding stoma.

In one or more embodiments, the holster is welded to the protection shield, such as plastic welding or thermo plastic welding. In one or more embodiments, the holster is welded to the protection shield, such as on the inside or outside of the first or second side of the protection shield.

In one or more embodiments, the holster is attached to an inside material of the protection shield.

In one or more embodiments, the holster is attached close to the outside circumferential of the protection shield, preferably on the side of the protective shield facing away from the skin, when the device is being used.

In one or more embodiments, the protective shield is combined and attached to each other parts being formed by the protective shield, the withdrawal sleeve and the insertion sleeve.

In one or more embodiments, the protection shield is circularly shaped.

In one or more embodiments, the protection shield has a diameter between 6 cm and 15 cm.

In one or more embodiments, the insertion and withdrawal system comprise a first and a second non-connected parts, wherein the first part is the protection shield interconnected with the holster and the second part is the ostomy device interconnected with the one or more withdrawal means.

In one or more embodiments, the insertion and withdrawal system comprise a first, a second, and a third non-connected parts, wherein the first part is the protection shield, the second part is the holster, and the third part is the ostomy device interconnected with the one or more withdrawal means.

The holster can also be a solitary part of the system. In this way the ostomy device is placed by another insertion mechanism than the holster and a protective shield is placed on to the stoma. When it is time to withdrawal, the protective shield is removed and a holster is placed around the stoma. This kind of holster may have side flanges to hold against the skin while withdrawing the ostomy device.

In one or more embodiments, the holster comprises side flanges configured to be compressed against the skin of the human being while withdrawing the ostomy device with the one or more withdrawal means.

Upon usage, the system is placed with the opening surrounding the stoma opening, with the stoma opening being able to protrude inside the protective shield. The ostomy device is inserted into the stoma opening guided by a finger or alike. Upon placement of the ostomy device into the stoma, the holster turns inside out, covering the finger or alike to protect it against contact with fecal secretion. The finger or alike could be brought approximately 2 to 10 cm into the stoma opening to place the ostomy device on the inside of the abdominal fascia; however, this distance can be above 10 cm in obese individuals. The finger or alike is removed and the ostomy device is in place with the withdrawal means coming out from the stoma opening. A thin layer of padding may then be placed on the inside of the holster in the stoma near the end, hereby ensuring that moist or secretion from the stoma are captured.

When the finger or alike is removed, if there is a cutting in the outside material of the protective shield, this cutting could be configured such that it will place itself like a thin invisible cut in the outside protective shield material. The outside protective shield material will help keep the folded holster in place inside the protective shield.

Upon withdrawal of the ostomy device, the withdrawal means are pulled and the ostomy device will come out through the stoma opening. The holster will unfold and cover the ostomy device upon withdrawal, ensuring that no fecal matter comes into contact with the outside of the holster. When the ostomy device is fully withdrawn into the holster, the protective shield is removed from the skin and discarded. A new ostomy device may then be placed in the stoma opening.

In one or more embodiments, the one or more withdrawal means are integrated in the holster or the protective shield.

In one or more embodiments, upon placement of the ostomy device in the ostomy, the sleeve turns inside out, covering the finger or alike to protect it against contact with faecal secretion.

In one or more embodiments, the finger or alike brought at least 2 cm into the stoma opening to place the ostomy device on the inside of the abdominal fascia.

In one or more embodiments, the finger or alike brought at least 2 cm into the stoma opening to place the ostomy device in the intestine on the inside of the abdominal fascia.

In one or more embodiments, a thin layer of padding is placed on the inside of the holster in a stoma near the end to ensure that moist or secretion from the stoma are captured.

In one or more embodiments, the device further comprises an insertion sleeve. In one or more embodiments, the insertion sleeve is part of the holster or interconnected with the holster. In this way the holster comprises or is attached to an invertible piece (the insertion sleeve). The insertion sleeve may have several functions. One, it protects and holds together the ostomy device before use (when in its inverted state). Two, it is flexible and soft, and hereby acts as a “finger glove” for insertion of the ostomy device, as discussed above. It hereby acts as a barrier between the finger and mucous membrane, so that the risk of infection or mechanical damage from e.g. a finger nail is minimised, as it would preferably only be the surface of the insertion sleeve that comes into contact with the mucosal membrane. Third, it breaks the contact between the mucous membrane, so flatus is avoided, by the invertible piece (the insertion sleeve) assuming the shape of the intestine and folds will appear lengthwise, creating low-pressure zones where the gas can pass unhindered, and without building up pressure. The gas will then flow towards one or more ventilation holes, and preferably passing through the carbon filter, if present, and finally out through the perforation in the protective shield.

Further, in one or more embodiments, the circumference and depth of the contact surfaces between the protective shield and the stoma are configured to be flexible and shape themselves naturally to non-uniform stomas. Ostomies have a large variation in shape and size, and therefore it is necessary that the product can adapt to the user to avoid discomfort during use and to ensure that the product closes tightly and that a potential filter does not clog.

When describing the embodiments, the combinations and permutations of all possible embodiments have not been explicitly described. Nevertheless, the mere fact that certain measures are recited in mutually different dependent claims or described in different embodiments does not indicate that a combination of these measures cannot be used to advantage. The present invention envisages all possible combinations and permutations of the described embodiments.
1. An insertion and withdrawal system for insertion of an ostomy device through an ostomy of a human being, subtracting the ostomy device, and containing the ostomy device after subtraction, the insertion and withdrawal system comprising:
   the ostomy device having an expandable elongated body comprising one or more absorbent materials;
   a protection shield having a first side and a second side;
   a holster attached to the protection shield, the holster configured to be in a folded state prior to insertion of the ostomy device through the ostomy of the human being; and
   one or more withdrawal means;
   wherein the first side of the protection shield has one or more attachment means for attaching the protection shield to the human being;
   wherein the one or more absorbent materials is configured to absorb intestinal fluid and waste inside the intestines of the human being;
   wherein the ostomy device is configured to be pulled out of the intestines of the human being by means of the one or more withdrawal means;
   wherein the holster is configured to transition to an unfolded state hereby covering the ostomy device when the ostomy device is pulled out of the intestines of the human being by means of the one or more withdrawal means; and
   wherein the holster in the unfolded state is further configured to contain the ostomy device after subtraction.
2. The insertion and withdrawal system according to any of the preceding items, wherein the string is in the form of a flat string, such as a flatwound string.
3. The insertion and withdrawal system according to any of the preceding items, wherein the expandable elongated body extends along a longitudinal direction of the ostomy device, the expandable elongated body being configured to maintain a compressed state, having a first diameter along the longitudinal direction, prior to insertion of the ostomy device through the ostomy of the human being and configured to expand radially to a non-compressed state, having a second diameter along the longitudinal direction, after insertion into the intestines of the human being through the ostomy, wherein the second diameter is larger than the first diameter.
4. The insertion and withdrawal system according to any of the preceding items, wherein the ostomy device is configured to absorb intestinal fluid and waste inside the intestines of the human being, the ostomy device comprising:
   an expandable elongated body extending along a longitudinal direction of the ostomy device and having a stoma end and an oral end spaced from the stoma end along the longitudinal direction, wherein the expandable elongated body comprises one or more absorbent materials; and
   one or more compression means configured to maintain the expandable elongated body in a compressed state, having a first diameter along the longitudinal direction, prior to insertion of the ostomy device through the ostomy of the human being;
   wherein the one or more withdrawal means are attached to the stoma end of the expandable elongated body;
   wherein the expandable elongated body is configured to expand radially to a non-compressed state, having a second diameter along the longitudinal direction, when released from the one or more compression means upon insertion into the intestines of the human being through the ostomy; and
   wherein the second diameter is larger than the first diameter.
5. The insertion and withdrawal system according to any of the preceding items, wherein the protection shield is a band aid or a patch.
6. The insertion and withdrawal system according to any of the preceding items, wherein the first side of the protection shield is configured to be in direct or indirect contact with the skin of the human being.
7. The insertion and withdrawal system according to any of the preceding items, wherein the protection shield has a through hole extending from the first side to the second side.
8. The insertion and withdrawal system according to item 7, wherein the holster is attached to the side of the through hole and harmonic folded.
9. The insertion and withdrawal system according to any of the preceding items, wherein the holster is attached to the second side of the protective shield and is harmonically folded from the outside towards the centre of the protective shield.
10. The insertion and withdrawal system according to any of items 7-9, wherein the through hole is circularly shaped.
11. The insertion and withdrawal system according to any one of items 7-10, wherein the holster is located around the through hole of the protection shield.
12. The insertion and withdrawal system according to any one of items 7-11, wherein the holster is accessible from the second side of the protection shield.
13. The insertion and withdrawal system according to any one of items 7-12, wherein the through hole has a diameter between 3 and 6 cm.
14. The insertion and withdrawal system according to any of the preceding items, wherein the holster is circumferentially folded in a harmonically shaped folding.
15. The insertion and withdrawal system according to any one of items 7-14, wherein the through hole is configured to allow the ostomy device to be inserted and withdrawn from the ostomy of the human being while the ostomy device is being covered by the holster.
16. The insertion and withdrawal system according to any of items 7-15, wherein the through hole comprises an insertion sleeve extending out and away from the through hole and the back side of the protective shield.
17. The insertion and withdrawal system according to any one of items 7-16, wherein the one or more attachment means for attaching the protection shield to the human being is an adhesive around the through hole to make the protection shield adhere to the skin of the human being.
18. The insertion and withdrawal system according to any one of items 7-17, wherein the ostomy device protrude through the through hole towards and past the first side of the protection shield while being covered by the holster on the second side of the protection shield.
19. The insertion and withdrawal system according to any of the preceding items, wherein the holster is attached to an insertion sleeve comprising the ostomy device on the second side of the protection shield prior to use.
20. The insertion and withdrawal system according to any of the preceding items, wherein the holster is both the insertion sleeve and the holster.
21. The insertion and withdrawal system according to any of the preceding items, wherein the system further comprises a carbon filter.
22. The insertion and withdrawal system according to any of the preceding items, wherein the protective shield comprises one or more ventilation holes.
23. The insertion and withdrawal system according to item 22, wherein the one or more ventilation holes is one or more perforations in the protective shield.
24. The insertion and withdrawal system according to any of the preceding items, wherein the protective shield comprises one or more integrated charcoal/carbon filters for odourless gas discharge.
25. The insertion and withdrawal system according to item 21 or 24, wherein the carbon filter is incorporated into the protective shield.
26. The insertion and withdrawal system according to any of the preceding items, wherein the system further comprises a bandage for collecting secretion from the ostomy.
27. The insertion and withdrawal system according to any of the preceding items, wherein the system further comprises one or more integrated moisture absorbent pads.
28. The insertion and withdrawal system according to any of the preceding items, wherein the holster is folded in a ring like manner in the folded state prior to insertion of the ostomy device through the ostomy of the human being.
29. The insertion and withdrawal system according to any of the preceding items, wherein the protection shield, the holster, and the one or more withdrawal means are interconnected parts attached to the ostomy device.
30. The insertion and withdrawal system according to any of the preceding items, wherein the holster is between 20 cm and 80 cm long in the unfolded state.
31. The insertion and withdrawal system according to any of the preceding items, wherein the holster has a diameter between 1.5 cm and 6 cm.
32. The insertion and withdrawal system according to any of the preceding items, wherein the protection shield is a flat bag having the first side being a backside or a skin side and the second side being an outside.
33. The insertion and withdrawal system according to any of the preceding items, wherein the second side is dome shaped hereby creating a cavity inside the protection shield between the first side and the second side to be able to contain a protruding stoma of the human being inside the cavity.
34. The insertion and withdrawal system according to any of the preceding items, wherein the one or more withdrawal means is one or more strings attached to the ostomy device and extending through the holster on the second side of the protection shield.
35. The insertion and withdrawal system according to any of the preceding items, wherein the one or more attachment means is attachment means configured to be attachable to a stoma baseplate.
36. The insertion and withdrawal system according to any of the preceding items, wherein the attachment means configured to be attachable to a stoma baseplate is a click system.
37. The insertion and withdrawal system according to any of the preceding items, wherein the holster is made of plastic, silicone, latex, or combinations hereof.
38. The insertion and withdrawal system according to any of the preceding items, wherein the holster is made of a highly elastic material, such as a thermoplastic elastomer (TPE) or silicone.
39. The insertion and withdrawal system according to any of the preceding items, wherein the holster is made of a highly elastic material having a very low Young's modulus and an ability to stretch more than 800% before breaking.
40. The insertion and withdrawal system according to any of the preceding items, wherein the holster is made of a highly elastic thermoplastic elastomer having an ability to stretch more than 800% before breaking.
41. The insertion and withdrawal system according to any of the preceding items, wherein the holster is made of a biodegradable material.
42. The insertion and withdrawal system according to any of the preceding items, wherein the holster has a material thickness of at least 500 microns, such as at least 600 microns, or such as around 700 microns.
43. The insertion and withdrawal system according to any of the preceding items, wherein the system further comprise a collar attached to the second side and wherein the holster is attached to the collar.
44. The insertion and withdrawal system according to item 43, wherein the holster is attached to the collar by connection means, such as elastic connection means.
45. The insertion and withdrawal system according to any of the preceding items, wherein the holster is welded to the protection shield, such as plastic welding or thermo plastic welding.
46. The insertion and withdrawal system according to any of the preceding items, wherein the holster is welded to the protection shield, such as on the inside or outside of the first or second side of the protection shield.
47. The insertion and withdrawal system according to any of the preceding items, wherein the holster is attached to an inside material of the protection shield.
48. The insertion and withdrawal system according to any of the preceding items, wherein the holster is attached close to the outside circumferential of the protection shield, preferably on the side of the protective shield facing away from the skin, when the device is being used.
49. The insertion and withdrawal system according to any of the preceding items, wherein the protective shield is combined and attached to each other parts being formed by the protective shield, the withdrawal sleeve, and the insertion sleeve.
50. The insertion and withdrawal system according to any of the preceding items, wherein the protection shield is circularly shaped.
51. The insertion and withdrawal system according to any of the preceding items, wherein the protection shield has a diameter between 6 cm and 15 cm.
52. The insertion and withdrawal system according to any of the preceding items, wherein the insertion and withdrawal system comprises a first and a second non-connected parts, wherein the first part is the protection shield interconnected with the holster and the second part is the ostomy device interconnected with the one or more withdrawal means.
53. The insertion and withdrawal system according to any of the preceding items, wherein the insertion and withdrawal system comprises a first, a second, and a third non-connected parts, wherein the first part is the protection shield, the second part is the holster, and the third part is the ostomy device interconnected with the one or more withdrawal means.
54. The insertion and withdrawal system according to any of the preceding items, wherein the holster comprises side flanges configured to be compressed against the skin of the human being while withdrawing the ostomy device with the one or more withdrawal means.
55. The insertion and withdrawal system according to any of the preceding items, wherein the device further comprises an insertion sleeve.
56. The insertion and withdrawal system according to item 55, wherein the insertion sleeve is part of the holster or interconnected with the holster.
57. The insertion and withdrawal system according to any of the preceding items, wherein the circumference and depth of the contact surfaces between the protective shield and the stoma are configured to be flexible and shape themselves naturally to non-uniform stomas.
58. A protection shield for use in an insertion and withdrawal system according to any of the preceding items, the protection shield having a first side and a second side.
59. A holster for use in an insertion and withdrawal system according to any of the preceding items, the holster being configured to be in a folded state prior to insertion of the ostomy device through the ostomy of the human being.
60. Use of an insertion and withdrawal system according to any one of items 1-57 for insertion of an ostomy device through an ostomy of a human being.
61. Use of an insertion and withdrawal system according to any one of items 1-57 for subtracting an ostomy device through an ostomy of a human being.
62. Use of an insertion and withdrawal system according to any one of items 1-57 for containing an ostomy device after subtraction of the ostomy device through the ostomy of a human being.
63. Use of a protection shield and/or a holster according to any one of items 1-57 for insertion of an ostomy device through an ostomy of a human being.
64. Use of a protection shield and/or a holster according to any one of items 1-57 for subtracting an ostomy device through an ostomy of a human being.
65. Use of a protection shield and/or a holster according to any one of items 1-57 for containing an ostomy device after subtraction of the ostomy device through the ostomy of a human being.
66. A method for insertion of an ostomy device through an ostomy of a human being, the method comprising the steps of:
   providing the insertion and withdrawal system according to any one of items 1-57;
   placing the system at an opening surrounding the stoma, with the stoma being able to protrude inside the protection shield;
   insert into the stoma opening, guided by a finger or alike, the ostomy device; and.
   removing the finger or alike and the ostomy device is in place with the one or more withdrawal means extending out from the stoma opening.
67. The method according to item 66, wherein upon placement of the ostomy device into the stoma opening, the sleeve turns inside out, covering the finger or alike to protect it against contact with fecal secretion.
68. The method according to any one of items 66-67, wherein the finger or alike brought at least 2 cm into the stoma opening to place the ostomy device in the intestine on the inside of the abdominal fascia.
69. The method according to any one of items 66-68, wherein a thin layer of padding is placed on the inside of the holster in a stoma near the end to ensure that moist or secretion from the stoma are captured.
70. A method for subtracting an ostomy device inserted through an ostomy of a human being, the method comprising the steps of:
   withdrawal of the ostomy device according to any one of items 1-57 by pulling in the one or more withdrawal means, such that the ostomy device is pulled out through the stoma opening; and.
   simultaneously unfolding the holster by pulling the one or more withdrawal means and the ostomy device, such as pulling through the through hole or opening of the protection shield.

The present disclosure will now be described with reference to the accompanying drawings, in which preferred example embodiments of the disclosure are shown. The disclosure may, however, be embodied in other forms and should not be construed as limited to the herein disclosed embodiments. The disclosed embodiments are provided to fully convey the scope of the disclosure to the skilled person.

### DETAILED DESCRIPTION OF THE DRAWINGS

The above objects, as well as additional objects, features and advantages of the present disclosure will be more fully appreciated by reference to the following illustrative and non-limiting detailed description of example embodiments of the present disclosure, when taken in conjunction with the accompanying drawings.

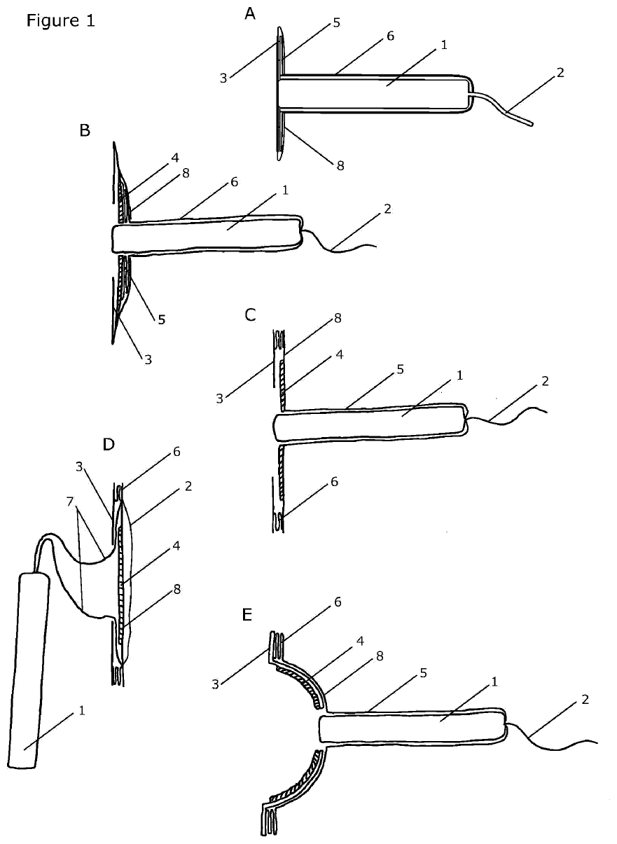

FIG. 1A shows the system according to one embodiment of the present disclosure. This figure illustrates the absorbent ostomy device 1 in a compressed state, attached to withdrawal means 2, placed inside, and attached to, the insertion sleeve 5. The insertion sleeve is interconnected with the withdrawal sleeve 6 (also called the holster), the latter in a folded configuration, placed in the protection shield 8. The end of the withdrawal sleeve 6 that is not attached to the insertion sleeve, is attached to the protection shield 8 around the through hole on the first side (the skin side) of the protection shield. The first side (skin side) of the protection shield having attachment means 3 for attaching the protection shield to the skin or a baseplate.

FIG. 1B shows the system according to another embodiment of the present disclosure. This figure illustrates the absorbent ostomy device 1 in a compressed state, attached to withdrawal means 2, placed inside, and attached to, the insertion sleeve 5. The insertion sleeve is interconnected with the withdrawal sleeve 6, the latter in a folded configuration, placed in the protection shield 8. The end of the withdrawal sleeve 6 that is not attached to the insertion sleeve, is attached to the inside of the second side of the protection shield 8. The first side of the protection shield having attachment means 3 for attaching the protection shield to the skin or a baseplate. Around the insertion hole of the absorbent ostomy device on the inside of the second side of the protection shield 8, is a padding 4.

FIG. 1C shows the system according to another embodiment of the present disclosure. This figure illustrates the absorbent ostomy device 1 in a compressed state, attached to withdrawal means 2, placed inside, and attached to, the insertion sleeve 5. The insertion sleeve is also attached to the second side of the protection shield 8. Between the first side (skin side) and the second side of the protection shield is the withdrawal sleeve 6 in a folded configuration, attached to each sides of the protection shield 8. The first side of the protection shield having attachment means 3 for attaching the protection shield to the skin or a baseplate. Around the insertion hole of the absorbent ostomy device on the inside of the second side of the protection shield 8, is a padding 4.

FIG. 1D shows the system according to another embodiment of the present disclosure. This figure illustrates the absorbent ostomy device 1 in a compressed state, attached to the protection shield 8 by an absorbent ostomy device string attachment 7. Inside the protection shield 8 between the first side (skin side) and the second side of the protection shield, is a padding 4. The outer side of the second side of the protection shield 8 has withdrawal means 2 attached. Between the first side (skin side) and the second side of the protection shield 8 is a withdrawal sleeve in a folded configuration, attached to each sides of the protection shield. The first side (skin side) of the protection shield having attachment means 3 for attaching the protection shield to the skin or a baseplate.

FIG. 1E shows the system according to another embodiment of the present disclosure. This figure illustrates the absorbent ostomy device 1 in a compressed state, attached to withdrawal means 2, placed inside, and attached to, the insertion sleeve 5. The insertion sleeve is interconnected with the withdrawal sleeve 6, the latter in a folded configuration, placed on, and attached to the flanges of a dome shaped protection shield 8. The flanges of the protection shield having attachment means 3 on the first side (skin side) for attaching the protection shield to the skin or a baseplate. Around the insertion hole of the absorbent ostomy device, on the inside of the second side of the protection shield 8, is a padding 4. The protection shield 8 is made from a material that makes it dome shaped and gives it some stability to protect a protruding stoma.

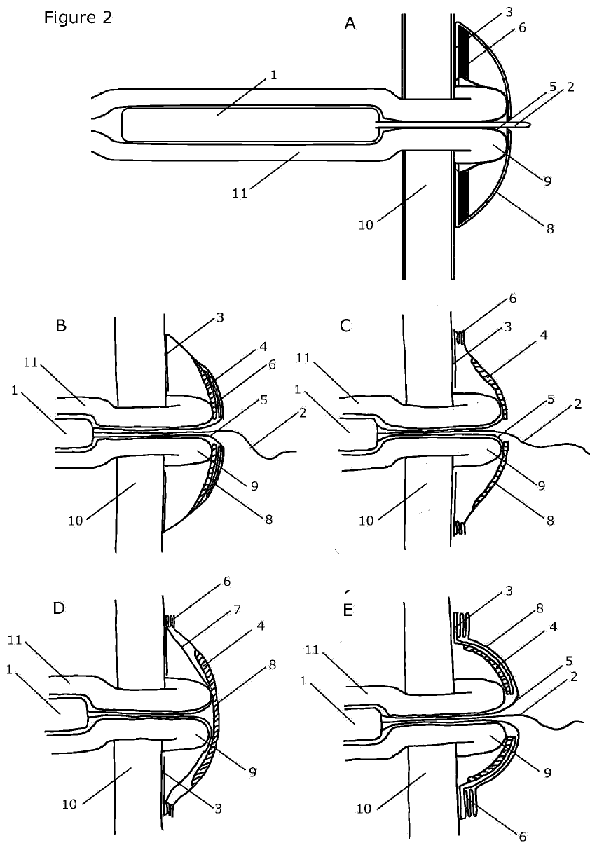

FIG. 2A shows the system according to one embodiment of the present disclosure. This figure illustrates the absorbent ostomy device 1 after insertion into the stoma 9, thereby placed in the intestine 11 on the inside of the abdominal wall 10. The absorbent ostomy device 1 is attached to withdrawal means 2 that runs from the absorbent ostomy device 1 out through the stoma 9 and through the insertion hole of the absorbent ostomy device in the second side of the protection shield 8 and ends on the outside of the second side of the protection shield. Attached to the absorbent ostomy device 1 is also an insertion sleeve 5 that surrounds the withdrawal means 2 running out through the stoma 9. The insertion sleeve 5 folds around and covers the stoma and is attached to a withdrawal sleeve 6, inside the protection shield 8. The withdrawal sleeve 6 is in a folded configuration placed on, and attached to, the inside of the first side (skin side) of the protection shield 8 in a circular manner around the through hole. The through hole in the first side (skin side) of the protection shield is placed around the stoma 9, while the second side of the protection shield 8 covers the stoma 9. The first side (skin side) of the protection shield 8 having an attachment means 3 for attaching the protection shield to the skin (or a baseplate).

FIG. 2B shows the system according to another embodiment of the present disclosure. This figure illustrates the absorbent ostomy device 1 after insertion into the stoma 9, thereby placed in the intestine 11 on the inside of the abdominal wall 10. The absorbent ostomy device 1 is attached to withdrawal means 2 that runs from the absorbent ostomy device 1 out through the stoma 9 and through the insertion hole of the absorbent ostomy device in the second side of the protection shield 8 and ends on the outside of the second side of the protection shield. Attached to the absorbent ostomy device 1 is also an insertion sleeve 5 that surrounds the withdrawal means 2 running out through the stoma 9. The other end of the insertion sleeve 5 is interconnected with the withdrawal sleeve 6, the latter in a folded configuration, placed on, and attached to the flanges of a dome shaped protection shield 8. Around the insertion hole of the absorbent ostomy device on the inside of the second side of the protection shield 8, is a padding 4. The through hole in the first side (skin side) of the protection shield 8 is placed around the stoma 9, while the second side of the protection shield 8 covers the stoma 9. The first side of the protection shield having attachment means 3 for attaching the protection shield to the skin (or a baseplate).

FIG. 2C shows the system according to another embodiment of the present disclosure. This figure illustrates the absorbent ostomy device 1 after insertion into the stoma 9, thereby placed in the intestine 11 on the inside of the abdominal wall 10. The absorbent ostomy device 1 is attached to withdrawal means 2 that runs from the absorbent ostomy device 1 out through the stoma 9 and through the insertion hole of the absorbent ostomy device in the second side of the protection shield 8 and ends on the outside of the second side of the protection shield. Attached to the absorbent ostomy device 1 is also an insertion sleeve 5 that surrounds the withdrawal means 2 running through the stoma 9. The other end of the insertion sleeve 5 is attached to the second side of the protection shield 8. Between the first side (skin side) and the second side of the protection shield is the withdrawal sleeve 6 in a folded configuration, attached to each sides of the protection shield 8. Around the insertion hole of the absorbent ostomy device on the inside of the second side of the protection shield 8, is a padding 4. The through hole in the first side (skin side) of the protection shield 8 is placed around the stoma 9, while the second side of the protection shield 8 covers the stoma 9. The first side of the protection shield having attachment means 3 for attaching the protection shield to the skin (or a baseplate).

FIG. 2D shows the system according to one embodiment of the present disclosure. This figure illustrates the absorbent ostomy device 1 after insertion into the stoma 9, thereby placed in the intestine 11 on the inside of the abdominal wall 10. The absorbent ostomy device 1 is attached to the protection shield 8 by an absorbent ostomy device string attachment 7. On the inside of the second side of the protection shield 8, is a padding 4 that covers the stoma 9. On the outer side of the second side of the protection shield 8 is attached withdrawal means 2. Between the first side (skin side) and the second side of the protection shield 8 is a withdrawal sleeve 6 in a folded configuration, attached to each sides of the protection shield 8. The first side of the protection shield having attachment means 3 for attaching the protection shield 8 to the skin (or a baseplate). The through hole in the first side (skin side) of the protection shield 8 is placed around the stoma 9, while the second side of the protection shield 8 covers the stoma 9. The first side of the protection shield having attachment means 3 for attaching the protection shield to the skin (or a baseplate).

FIG. 2E shows the system according to another embodiment of the present disclosure. This figure illustrates the absorbent ostomy device 1 after insertion into the stoma 9, thereby placed in the intestine 11 on the inside of the abdominal wall 10. The absorbent ostomy device 1 is attached to withdrawal means 2 that runs from the absorbent ostomy device 1 out through the stoma 9 and through the insertion hole of the absorbent ostomy device in the second side of the protection shield 8 and ends on the outside of the second side of the protection shield. Attached to the absorbent ostomy device 1 is also an insertion sleeve 5 that surrounds the withdrawal means 2 running out through the stoma 9. The insertion sleeve is interconnected with the withdrawal sleeve 6, the latter in a folded configuration, placed on, and attached to the flanges of the second side of the dome shaped protection shield 8. The flanges of the protection shield having attachment means 3 on the first side (skin side) for attaching the protection shield to the skin (or a baseplate). Around the insertion hole of the absorbent ostomy device, on the inside of the second side of the protection shield 8, is a padding 4. The flanges of the protection shield having attachment means 3 on the first side (skin side) for attaching the protection shield to the skin or a baseplate. Around the insertion hole of the absorbent ostomy device, on the inside of the second side of the protection shield 8, is a padding 4. The protection shield 8 is in a material that makes it dome shaped and gives it some stability to protect a protruding stoma. The through hole in the first side (skin side) of the protection shield 8 is placed around the stoma 9, while the second side of the protection shield 8 protrudes around the stoma 9.

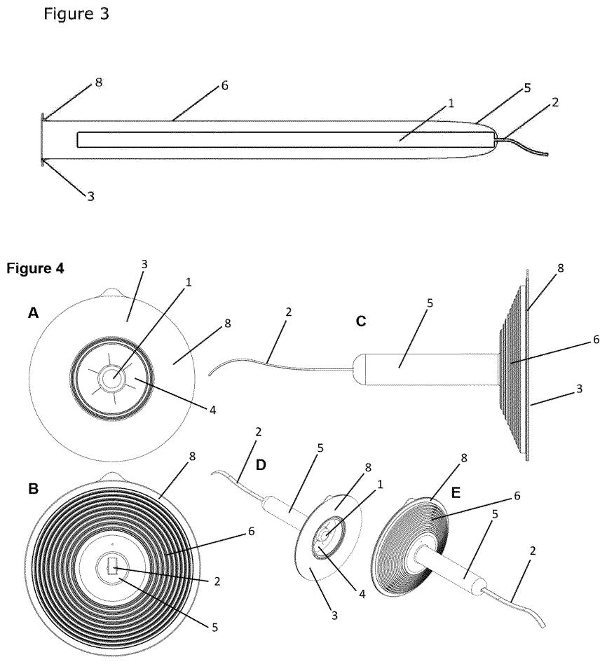

FIG. 3 shows the insertion and withdrawal system of the absorbent ostomy device after withdrawal according to multiple embodiments, when the device has been withdrawn. The elongated absorbent ostomy device 1 is shown in a non-compressed state after absorption of intestinal fluid. The absorbent ostomy device 1 is withdrawn in an unfolded withdrawal sleeve 6 attached to the absorbent ostomy device 1 in one end and to the protection shield 8 in the other end.

FIG. 4A-E shows the system according to one embodiment of the present disclosure. FIGS. 4A-E show different views of the system, such as front view (B), back view (A), side view (C) and two other perspectives (D and E). These figures illustrate the absorbent ostomy device 1 in a compressed state, attached to withdrawal means 2, placed inside, and attached to, the insertion sleeve 5. The insertion sleeve is interconnected with the withdrawal sleeve 6 (also called the holster), the latter in a harmonically folded configuration, placed on one side of the protection shield 8. The end of the withdrawal sleeve 6 that is not attached to the insertion sleeve 5, is attached to the protection shield 8 close to the outside circumferential of the protection shield 8. The protection shield 8 comprises a through hole on the first side (the skin side) of the protection shield 8. The first side (skin side) of the protection shield 8 having attachment means 3, such as an adhesive, for attaching the protection shield to the skin or a baseplate. The system is placed over the stoma of the user, and the insertion sleeve 5 is used as a “glove” to insert the ostomy device 1 through the stoma. The harmonic folded withdrawal sleeve 6 and the insertion sleeve 5, may preferably be formed from one single piece of material, thereby having no seam at the border between the two sleeves 5 and 6. Alternatively, they may be attached to each other via e.g. thermo plastic welding or other types of strong adhesion. The figures, especially A and D show how padding 4 is positioned around the insertion hole of the absorbent ostomy device on the inside of the second side of the protection shield 8. In the embodiment it is further shown, how the withdrawal means 2 is in the form of a flatwound string, which can be made of any material suitable for the use of withdrawing the ostomy device. The string is potentially expanding throughout the full length of the ostomy device and mat preferably be made with integrated rubber bands to make the ostomy device flexible in length. Further, it can be observed how the through hole of the protective shield 8 together with the harmonically folded withdrawal sleeve 6 forms a dome-like shape, which has the ability to protect and stabilize a protruding stoma.

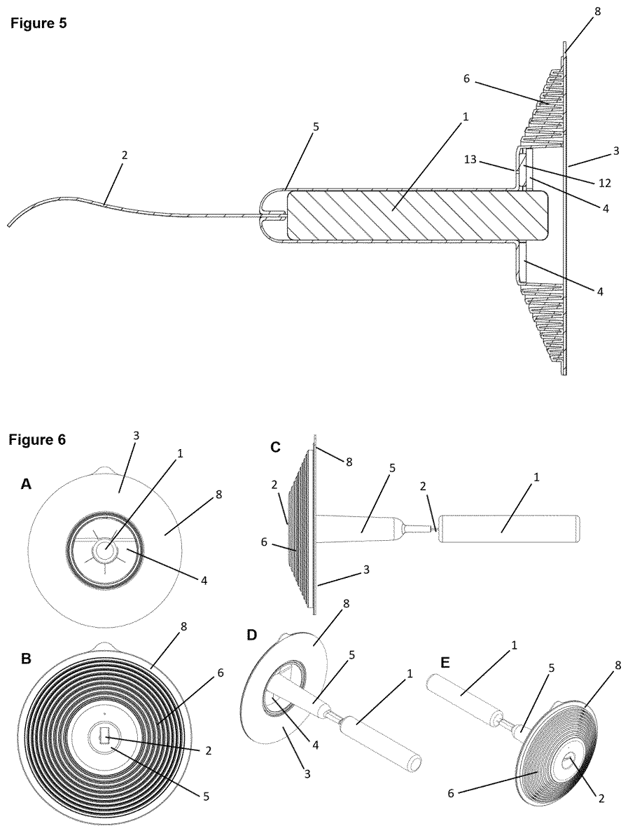

FIG. 5 is a cut-through down the middle of the side view of FIG. 4C. Besides what has already been disclosed in regards to FIG. 4, it is here clearly shown how the withdrawal sleeve 6 is harmonically folded and attached to the outer circumference of the protective shield 8. The withdrawal sleeve 6 is gradually extending further and further away from the side of the protective shield 8 not facing the skin, hereby also forming the dome shape of the system.

Alternatively, to a gradually extending shape, as disclosed above, the withdrawal sleeve 6 may comprise any suitable shape and folding mechanism depending on the given purpose and design. One alternative could e.g. be a withdrawal sleeve 6 that is overall flat and thereby not extending further and further away from the side of the protective shield 8 not facing the skin, hereby forming a substantially flat surface facing away from the skin side, which may e.g. have a visual appeal to some users.

It is further shown how the withdrawal sleeve 6 is interconnected with the insertion sleeve 5 by being formed of one piece of material. Further shown is how the padding 4 is positioned inside the through hole of the protective shield 8, and how a filter 12, such as a carbon filter is positioned between the padding 4 and a ventilation hole 13 in the form of a perforation in the withdrawal and/or insertion sleeve 6 and 5. The protective shield 8 may be seen as just the protective shield 8, or as the combined and attached to each other parts, being formed by the protective shield 8, the withdrawal sleeve 6, and the insertion sleeve 5.

FIG. 6A-E shows the system according to one embodiment of the present disclosure. This figure illustrates the absorbent ostomy device 1 after insertion into the stoma (not shown for simplicity), thereby placed in the intestine on the inside of the abdominal wall. The absorbent ostomy device 1 is attached to withdrawal means 2 that runs from the absorbent ostomy device 1 out through the stoma and through the insertion hole of the absorbent ostomy device in the second side of the protection shield 8 and ends on the outside of the second side of the protection shield. The withdrawal sleeve 6 is in a harmonically folded configuration placed on, and attached to the protection shield 8 in a circular manner around the through hole. The through hole in the first side (skin side) of the protection shield is placed around the stoma, while the second side of the protection shield 8, here being the harmonically folded withdrawal sleeve 6, covers the stoma. The first side (skin side) of the protection shield 8 having an attachment means 3 for attaching the protection shield to the skin (or a baseplate). Attached to the withdrawal sleeve 6 is also an insertion sleeve 5 that surrounds the withdrawal means 2 running out through the stoma. Besides the ostomy device 1 being inserted into the stoma, the insertion sleeve 5 will also be pushed into the stoma by the user's finger. In this way, the insertion sleeve 5 serves the function of also being a finger glove for the user, and may therefore also be referred to as a finger glove. The insertion sleeve 5 is here shown as a seamless part of the withdrawal sleeve 6, however it may also just be provided as an interconnected part of the withdrawal sleeve 6. By comparing FIGS. 4 and 6, it can be seen that the insertion sleeve 5 is an invertible piece. The insertion sleeve 5 hereby has several functions as discussed throughout the description.

Further shown is how a padding 4 is positioned around the insertion hole of the absorbent ostomy device on the inside of the second side of the protection shield 8. In the embodiment is further shown, how the withdrawal means 2 is in the form of a flatwound string, which can be made of any material suitable for the use of withdrawing the ostomy device. Further, it can be observed how the through hole of the protective shield 8 together with the harmonic folded withdrawal sleeve 6 forms a dome-like shape, which has the ability to protect and stabilize a protruding stoma, also after the insertion sleeve 5 has entered the stoma.

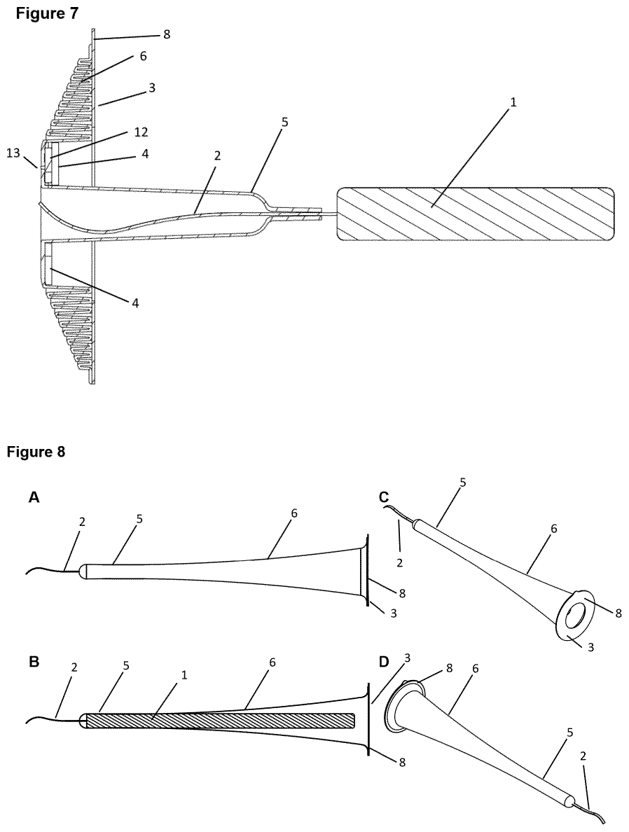

FIG. 7 is a cut-through down the middle of the side view of FIG. 6C. Besides what has already been disclosed in regards to FIG. 6, it is here clearly shown how the withdrawal sleeve 6 is harmonic folded and attached to the outer circumferential of the protective shield 8. The withdrawal sleeve 6 is gradually extending further and further away from the side of the protective shield 8 not facing the skin, hereby also forming the dome-shaped of the system. It is further shown how the withdrawal sleeve 6 is interconnected with the insertion sleeve 5 by being formed of one piece of material. It is also shown how the insertion sleeve 5 has been inverted and is now facing the opposite direction compared to prior to insertion (FIG. 5). Further shown is how the padding 4 is positioned inside the through hole of the protective shield 8, and how a filter 12, such as a carbon filter is positioned between the padding 4 and a ventilation hole 13 in the form of a perforation in the withdrawal and/or insertion sleeve 6 and 5. The protective shield 8 may be seen as just the protective shield 8, or as the combined and attached to each other parts, being formed by the protective shield 8, the withdrawal sleeve 6 and the insertion sleeve 5.

FIG. 8 shows the insertion and withdrawal system of the absorbent ostomy device after withdrawal according to multiple embodiments, when the device has been withdrawn. A-D shows various views of the embodiment. The elongated absorbent ostomy device 1 is shown in a non-compressed state after absorption of intestinal fluid. The absorbent ostomy device 1 is withdrawn in an unfolded withdrawal sleeve 6 attached to the absorbent ostomy device 1 in one end and to the protection shield 8 in the other end. In multiple embodiments, as also shown here, the withdrawal sleeve 6 is attached to, or comprise, an insertion sleeve 5. The figure shows how the withdrawal sleeve 6 and the insertion sleeve 5 are seamlessly bounded together, preferably by making them from one piece of material without the need for adhesion, such as welding. The protective shield 8, if provided with a good adhesive, can after withdrawal of the ostomy device 1 be closed, by folding the protective shield 8 at the middle, thereby taking advantage of the adhesive, which will then close the protective shield, and the used insertion and withdrawal system can be discarded without any leakage or smell from the system after closure.

### REFERENCES

1—Absorbing ostomy device
2—Withdrawal means
3—Skin attachment means
4—Padding
5—Insertion sleeve
6—Withdrawal sleeve/Holster
7—Absorbing ostomy device string attachment
8—Protective shield
9—Stoma
10—Abdominal wall
11—Intestinal wall
12—Filter
13—Ventilation hole

1. An insertion and withdrawal system for insertion of an ostomy device through an ostomy of a human being, subtracting the ostomy device, and containing the ostomy device after subtraction, the insertion and withdrawal system comprising:
an ostomy device comprising an expandable elongated body comprising an absorbent material ;
a protection shield comprising a first side and a second side;
a holster attached to the protection shield, the holster configured to be in a folded state prior to insertion of the ostomy device through an ostomy of a human being; and
a withdrawer;
wherein the first side of the protection shield comprises an attachment for attaching the protection shield to the human being;
wherein the absorbent material is configured to absorb intestinal fluid and waste inside an intestine of the human being;
wherein the ostomy device is configured to be pulled out of the intestine of the human being by the withdrawer;
wherein the holster is configured to transition to an unfolded state hereby covering the ostomy device when the ostomy device is pulled out of the intestine of the human being by the withdrawer; and
wherein the holster in the unfolded state is further configured to contain the ostomy device after subtraction.

2. The insertion and withdrawal system according to claim 1, wherein the protection shield has a through hole extending from the first side to the second side.

3. The insertion and withdrawal system according to claim 2, wherein the holster is located in the through hole of the protection shield.

4. The insertion and withdrawal system according to claim 2, wherein the holster is accessible from the second side of the protection shield.

5. The insertion and withdrawal system according to claim 2, wherein the though hole is configured to allow the ostomy device to be inserted and withdrawn from the ostomy of the human being while the ostomy device is being covered by the holster.

6. The insertion and withdrawal system according to claim 1 wherein the holster is folded in a ring like manner in the folded state prior to insertion of the ostomy device through the ostomy of the human being.

7. The insertion and withdrawal system according to claim 1, wherein the protection shield, the holster, and the withdrawer are interconnected parts attached to the ostomy device.

8. The insertion and withdrawal system according to claim 1, wherein the holster is between 20 cm and 60 cm long in the unfolded state.

9. The insertion and withdrawal system according to claim 1, wherein the insertion and withdrawal system comprises a first non-connected part, a second non-connected part, and a third non-connected part, wherein the first non-connected part is the protection shield, the second non-connected part is the holster, and the third non-connected part is the ostomy device interconnected with the withdrawer

10. (canceled)

11. (canceled)

12. A protection shield for use in an insertion and withdrawal system, the protection shield comprising:
a first side having a throughhole and the first side comprising an attachment;
a second side having the throughhole, wherein the throughhole has a diameter of between 3 and 6 cm.

13. The protection shield according to claim 12, wherein the protection shield is circularly shaped.

14. The protection shield according to claim 13, wherein the protection shield has a diameter of between 6 cm and 15 cm.

15. A holster for use in an insertion and withdrawal system, the holster comprising a structure that has a folded state and an unfolded state, wherein the structure in the folded state has an annular shape with an outer diameter of between 1.5 cm and 6 cm.

16. The holster according to claim 15, wherein the structure is made of a highly elastic material having a Young's modulus at or below 5 MPa.

17. The holster according to claim 15, wherein the structure has a material thickness of at least 500 microns.

18. The holster according to claim 15, wherein the structure in the folded state comprises a first annular fold and a second annular fold that is concentric with the first annular fold and has a different outer diameter than that of the first annular fold.

19. The holster according to claim 15, wherein the structure in the unfolded state comprises a conical-like shape.

20. The holster according to claim 18, wherein the structure in the unfolded state comprises a conical-like shape.

21. A protection shield and holster system comprising:
a protection shield comprising
a first side having a throughhole and the first side comprising an attachment; and
a second side having the throughhole; and
a holster attached to the protection shield, the holster comprising:
a structure comprising a folded state and an unfolded state, wherein the throughhole passes through the structure.

22. The protection shield and holster system according to claim 21, wherein the throughhole has a diameter of between 3 and 6 cm.

23. The protection shield and holster system according to claim 21, wherein the protection shield is circularly shaped.

24. The protection shield and holster system according to claim 23, wherein the protection shield has a diameter of between 6 cm and 15 cm.

25. The protection shield and holster system according to claim 21, wherein the structure in the folded state has an annular shape with an outer diameter of between 1.5 cm and 6 cm.

26. The protection shield and holster system according to claim 21, wherein the structure is made of a highly elastic material having a Young's modulus at or below 5 MPa.

27. The protection shield and holster system according to claim 21, wherein the structure has a material thickness of at least 500 microns.

28. The protection shield and holster system according to claim 21, wherein the structure in the folded state comprises a first annular fold and a second annular fold that is concentric with the first annular fold and has a different outer diameter than that of the first annular fold.

29. The protection shield and holster system according to claim 21, wherein the structure in the unfolded state comprises a conical-like shape.

30. The protection shield and holster according to claim 28, wherein the structure in the unfolded state comprises a conical-like shape.
